(19) United States
(12) Patent Application Publication
(10) Pub. No.: US 2004/0127809 A1
(43) Pub. Date: Jul. 1, 2004

(54) Device for determining acoustically evoked brain potentials

(76) Inventor: Wolfgang Kopke, Berlin (DE)

Correspondence Address: Dykema Gossett, Suite 300 West, 1300 I Street NW, Washington, DC, 20005-3306, US

(21) Appl. No.: 10/471,058

(22) PCT Filed: Mar. 7, 2002
(86) PCT No.: PCT/EP02/02534

(30) Foreign Application Priority Data
Mar. 9, 2001 (EP) 01610023.2

(51) Int. Cl. A61B 5/04

(52) U.S. Cl. 600/544; 600/559

(57) ABSTRACT

The invention relates to a device for determining acoustically evoked brain potentials in brainstem audiometry from electrodes applied to the head of a subject, where the device comprises a plurality of electrodes, i.e. at least one pickup electrode and a reference electrode, to be applied at different points of the head, where the device comprises means for determining the impedance between the electrodes and the head as well as means for visually indicating a level of impedance or delivering this information to the connected audiometer.

### BACKGROUND OF THE INVENTION

The derivation of acoustically evoked electrical brain potentials of a subject is a known audiometric diagnostic method for testing hearing and for evaluating various causes of hearing damage without the active participation of the subject.

This method is referred to in the field as ERA (electric response audiometry) or BERA (brainstem electric response audiometry) or brainstem audiometry. Areas of application for this method include for example the performance of the first hearing tests in newborns, testing the hearing of infants or of unconscious persons such as accident victims for example, and the diagnosis of neurologic diseases, for example neurinomas of the acoustic nerve. Intraoperative hearing tests are also possible with this method.

Electrical brain potentials are triggered by acoustic stimulation of the ear with conduction through air or bone. Headphones are usually used for the purpose. The electrical signals that are thus generated by the brainstem are picked up by electrodes applied to the head. Usually three electrodes are used, namely one electrode to determine the reference potential and two active electrodes to derive the acoustically-evoked electrical signals at two different locations on the head.

Acoustic stimulation of the ear can take for example the form of click stimuli or, for direct determination of the hearing threshold, of a rapid sequence of clicks with increasing volume. Other types of stimuli are of course also possible. The brainstem generates potential waves at each click, which are averaged after being picked up and conducted away by the electrodes.

In the previously known devices the measuring results are relying on a constantly good electrical connection between the head and the electrode, resulting in low impedance. In practise this is however nor always a simple task. The measurements may often be repeated due to poor measuring results, which are not detected during measurement. There is for this reason a need for improvement of the previously known devices of this type.

It is therefore an object of the invention to provide a device to permit simpler and easier use and hence achievement of better results while performing brainstem audiometry.

### SUMMARY OF THE INVENTION

This object is achieved by the device described in claim 1.

By providing a visual indication of the impedance level there is a possibility of checking this during measurement and hence provide an immediate correction to the positioning of the electrode so as to achieve the desired low impedance.

In a preferred embodiment the means for visually indicating a level of impedance are located on the structure holding the electrodes, or a preamplifier where the electrodes are connected via cables. This allows for a simultaneous visual contact with the structure during a correction operation and the indicator means.

Preferably the means for determining the impedance are integrated with the structure or the device. This also improves the operation and visibility of the indicator means.

In a preferred embodiment the means for determining the impedance comprises switching arrangements for each electrode enabling the switching of each electrode into a impedance measuring mode and back to normal brain potential measuring mode. This provides for a possibility of obtaining a reliable measurement of the impedance without disturbing the measurement.

In one preferred embodiment the means for visually indicating a level of impedance comprises a single light emitter, e.g. a diode, indicating either a low or a high impedance level.

In another preferred embodiment the means for visually indicating a level of impedance comprises two light emitters, e.g. diodes, where one is indicating a low impedance level and the other is indicating a high impedance level.

In an device according to the invention, the electrode unit can also include an electroencephalograph (“EEG”) amplifier as a component, so that a minimum conduction path is provided between the pickup electrodes and the EEG amplifier, and thus the possibility of stray potentials being picked up is minimized.

### BRIEF DESCRIPTION OF THE DRAWINGS

FIG. 1 shows a schematic circuit forming part of the device according to the invention,

FIG. 2 shows a top view of a part of an example of a device according to the invention,

FIG. 3 shows a bottom view of a part of a device according to the invention,

FIG. 4 shows a side view of a part of a device according to the invention.

### DETAILED DESCRIPTION OF THE INVENTION

The device according to the invention will now be described briefly in terms of its important details, with reference to the embodiments shown in the enclosed drawings.

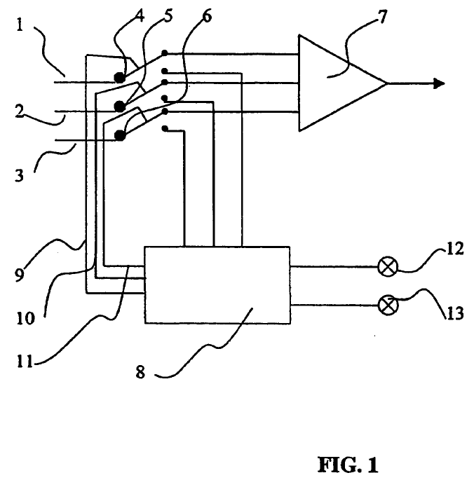

From FIG. 1 a schematic diagram appears. The diagram shows three inputs 1,2,3 each leading to a switch 4,5,6. The switches are switching between a measuring mode and a test mode. In the measuring mode the input signals are transmitted to an EEG amplifier 7 and further to an audiometer used in a measuring process. In the test mode the impedance is measured between the electrode and the skin of the individual on which the electrodes are placed. The test of the impedance is carried out with a predetermined sampling frequency and is controlled by the control electronics 8 adapted to control the switches via connections 9,10,11. The result of the impedance measurement is indicated by the diodes 12,13, where one indicated a too high level of the impedance and the other one indicated a satisfactory low level of the impedance.

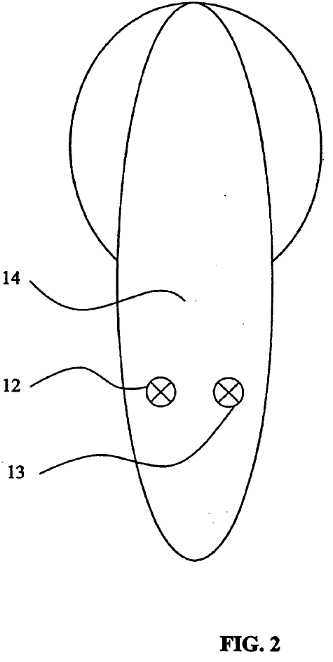

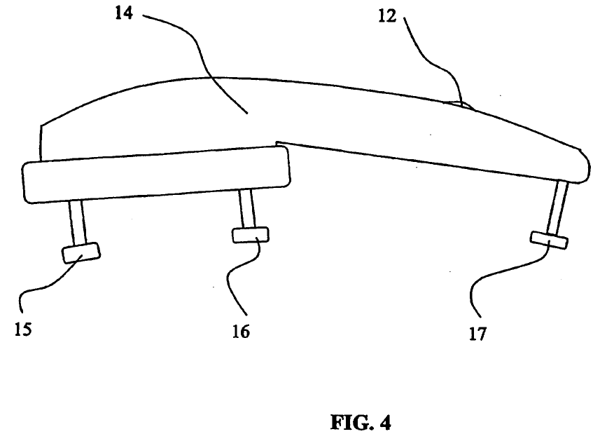

The device according to FIGS. 2, 3 and 4 consists of a housing 14 with a plurality of arms that have electrodes 15,16,17 at their ends, a earphone 18 integrated in the housing. An EEG amplifier (not shown) forms part of the device.

A cable (not shown) connects the device with the rest of the audiometer used for brainstem audiometry, said audiometer generating the signals for acoustic stimulation of the ear and processing and evaluating the derived brainstem potentials. In the embodiment, a single cable is shown that can contain both a line to supply electrical click signals and also a line to conduct the preamplified brainstem potentials from EEG amplifier. Of course, separate cables or wireless transmission pathways can also be used for the purpose.

The electrical potentials generated in the brainstem by acoustic stimulation of the ear are picked up by the electrodes on the arms. Usually three electrodes are used, namely a reference electrode for detecting a reference potential and two pickup electrodes. The reference electrode is brought into contact with the head in front of the ear, and one of the two deriving electrodes is placed behind the ear and the other in the area of the crown of the head.

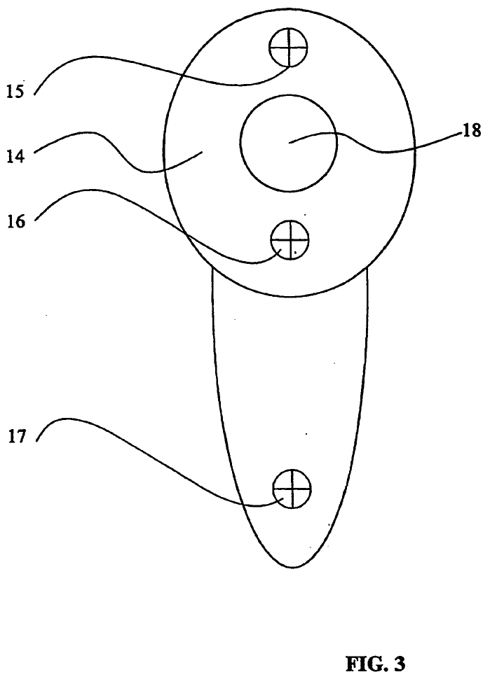

In the embodiment according to FIG. 3, electrode 17 is the pickup electrode that detects brainstem potentials in the vicinity of the crown of the head and electrode 16 is the reference electrode. The second pickup electrode 15 is applied to the head behind the ear.

1. A device for use in determining acoustically evoked brain potentials in brainstem audiometry from electrodes applied to the head of a subject, the device comprising: a plurality of electrodes comprising at least one pickup electrode and a reference electrode, to be applied at different points of the head, where the device comprises means for determining the impedance between the electrodes and the head as well as means for visually indicating a level of impedance, where the electrodes are mounted on a structure and where the means for visually indicating a level of impedance are located on the structure holding the electrodes.

2. A device according to claim 1, where the means for visually indicating a level of impedance are located on a preamplifier where the electrodes are connected via cables.

3. A device according to claim 1 or 2, where the means for determining the impedance are integrated with the device, preferably a structure holding the electrodes.

4. A device according to any of the preceding claim, where the means for determining the impedance comprises switching arrangements for each electrode enabling the switching of each electrode into a impedance measuring mode and back to normal brain potential measuring mode.

5. A device according to any of the preceding claims, where the means for visually indicating a level of impedance comprises a single light emitter, e.g. a diode, indicating either a low or a high impedance level.

6. A device according to any of the preceding claims, where the means for visually indicating a level of impedance comprises two light emitters, e.g. diodes, where one is indicating a low impedance level and the other is indicating a high impedance level.

7. A device according to any of the preceding claims, where the means for information the level of the of impedance is send via a cable ore wireless to the audiometer.
